(19) United States
(12) Patent Application Publication
(10) Pub. No.: US 2008/0051555 A1
(43) Pub. Date: Feb. 28, 2008

(54) Support Having Affinity for Antibody

(75) Inventors: Masahiro Iwakura, Ibaraki (JP); Kiyonori Hirota, Ibaraki (JP); Hiroyuki Sota, Ibaraki (JP)

Correspondence Address: MORGAN LEWIS & BOCKIUS LLP, 1111 PENNSYLVANIA AVENUE NW, WASHINGTON, DC, 20004, US

(73) Assignee: NATIONAL INSTITUTE OF ADVANCED INDUSTRIAL SCIENCE, Ibaraki (JP)

(21) Appl. No.: 10/575,254

(22) PCT Filed: Oct. 7, 2004
(86) PCT No.: PCT/JP04/14828

§ 371 Date: May 8, 2007

(30) Foreign Application Priority Data
Oct. 10, 2003 (JP) 2003-352937

(51) Int. Cl. C07K 1/14; C07K 14/00

(52) U.S. Cl. 530/300; 530/350; 530/387.1; 530/412

(57) ABSTRACT

A support having an affinity for an antibody characterized in that the carboxy end of a protein or a peptide, which is capable of binding to an antibody molecule, is immobilized via an amide bond mediated by a linker sequence to an insoluble support having a primary amino group. This support has an excellent ability to adsorb antibody molecules.

### TECHNICAL FIELD

The present invention relates to a support to which a protein having a specific affinity for an antibody is immobilized, a modified protein binding to an antibody to be used for the immobilization, a method of separating and purifying an antibody by using the immobilization support and so on.

### BACKGROUND ART

Since an antibody molecule has a highly-selective binding property to a specific molecule serving as an antigen thereof, methods of detecting specific molecules in biochemical samples by applying this excellent characteristic have been widely performed not only for laboratory uses but also in clinical examinations. Moreover, medical applications have been vigorously attempted with employing antibody molecules themselves as medicinal preparations, and antibody molecules have been widely employed both scientifically and industrially as highly useful molecules among molecules with biologic origins.

These antibody molecules are produced in the blood of laboratory animals such as humans, mouse, rat, rabbit or sheep, which have been dosed with an antigen, and they are acquired by purifying a serum fraction obtained from the blood then collected. In addition, the technique of producing a monoclonal antibody, which was developed in 1970's, enabled the continuous production of antibody molecules by culturing antibody-producing cells having been established in vitro. According to this method of producing industrially useful antibody molecules, antibody molecules are obtained by the purification from the obtained liquid culture medium.

As the methods of purifying an antibody, there has been required a procedure of efficiently purifying the target antibody molecules alone from a large amount of mixed non-objective biologic molecules in a serum sample in the case of originating in a laboratory animal blood as described above or in a culture supernatant in the case of originating in antibody-producing cells. Since the liquid chromatography method shows a favorable separation ability, an excellent performance and no invasiveness to target molecules, it has been frequently employed therefor. As mentioned above, when antibody molecules are used as a specific detection means, the purity of the antibody molecules itself is important. Moreover, when antibody molecules are used for medical purposes, contamination with residual matters is seemingly a serious problem, since it not only brings about lowering in the drug effect but also accompanies toxicity in some cases. The liquid chromatography method is a technique which includes packing a column with insoluble fine particles called a support, passing a liquid sample through the layer packed with the support and thereby separating molecules in the sample via interactions with the solid surface. Ion exchange chromatography in which target antibody molecules are separated in accordance with the charge thereof depending on the difference in the physical and/or chemical properties of the support surface, hydrophobic chromatography utilizing the difference in hydrophobic natures and gel filtration chromatography in which separation is conducted depending on the difference in molecular weights have been employed in practice for a long time. At the early stage, these techniques were employed as means of purifying antibody molecules.

However, operation conditions for achieving a satisfactory degree of purification (purity) can be hardly found out in these techniques and several different column operations should be employed to complete the purification.

In order to overcome the difficulties in these traditional chromatographic techniques and to achieve a high degree of purification by a single-stage chromatographic operation, affinity chromatography has been established. According to the affinity chromatography, the molecule having a specific ability to bind to the target molecule are selected as binding ligand and provided on the support surface. This ligand is capable of strongly binding only to the target molecule. When the liquid sample is passed through the support, then only the target molecules are captured by the surface while other non-target molecules pass through as they are. The target molecules thus captured are collected by elution. Owing to the strict molecular differentiation, much high degree of purification can be achieved in comparison with the traditional chromatographic methods. Attempts have been made to employ various molecules as ligands, such as an antibody molecule in the case that the target molecule is an antigen, lectin (a sugar-binding protein) for a glycoprotein, a substrate analog for an enzyme, other low-molecular weight compounds (a dye, a hapten, an inhibitor molecule) having been confirmed as being capable to binding to specific proteins.

Although there is a great demand in practice for the application of affinity chromatography to the purification of antibody molecules, there are some technical difficulties, and therefore, studies have been promptly made thereon. Consequently, it has been found that natural antibody-binding proteins typified by protein A enable the application (see Non-Patent Document 1).

Protein A, which is a protein to be present as a cell wall constituent of Staphylococcus aureus, is capable of strongly binding to the Fc (constant) region of an antibody molecule. Differing from the Fab (variable) region participating in binding to an antigen, a structure common to various classes/subclasses of antibody molecules has been conserved in the Fc region. Thus, it has been attempted to employ protein A as a ligand in affinity chromatography as an antibody-binding molecule usable in common to various antibody molecules having different antigenicities. Namely, this method includes immobilizing protein A, which is a protein, to the surface of the support and allowing the protein to interact with a sample solution containing a target antibody molecule, thereby conducting purification (see Patent Document 1).

It appears that the ideal support to be used in affinity chromatography is one having both of the following two characteristics, i.e., 1) ligand molecules being stably sustained on the support during the chromatographic operation or storage; and 2) having a large adsorption capacity for the target molecule per unit volume of the support. The characteristic 1) as described above mainly affects the reproducibility in the affinity chromatographic operation with the use of the support and the operation conditions such as solvent and temperature to be employed. The characteristic 2), which affects the performance of the affinity chromatography support it self, is a highly important factor from the industrial viewpoint since the productivity and economic efficiency depend on this characteristic. Moreover, these two characteristics affect each other as, for example, the lack of the characteristic 1) (i.e., unstable binding of the ligand molecule) would cause lowering in the characteristic 2) (i.e., decrease in the effective adsorption capacity with the passage of time due to the drop out of ligand).

Concerning affinity chromatography using protein A as the ligand, the special attention was paid to the characteristic 1) in Patent Document 1 as cited above and an agarose support having been activated with cyanogen bromide (CNBr) was treated with protein A to attach the ligand via a strong covalent bond. Since it had been a practice before that to immobilize a protein by forming a bond via physical adsorption with the use of, for example, the own charge of the protein, the above-described method is advantageous in that a bound state with an extremely higher stability can be established thereby. According to this method, on the other hand, binding to primary amino groups distributed in the immobilized protein A is utilized, and therefore, it is not possible to control the binding sites between the support and protein A. Accordingly, there arises a problem in which the immobilized protein A molecules are oriented at random on the support and the sites essentially required in the binding activity are not exposed in the solvent side, or these sites themselves are utilized in the binding, thus the apparent activity with respect to the amount of the bound protein A is lowered.

Furthermore, there has been pointed out another problem in which, owing to carrying multiple primary amino groups, protein A binds at multiple sites and thus brings about structural restriction, which causes the inactivation of the protein. Although the characteristic 1) is taken into consideration in the invention as described in Patent Document 1, it suffers from a serious problem concerning the characteristic 2). As discussed above, affinity chromatography using protein A is an industrially important technique whereby an antibody molecule highly important as a drug can be efficiently purified. From the viewpoint of industrial application, there is another important characteristic in addition to the characteristics 1) and 2) as described above. That is to say, such a support itself should be subjected to sterilization and washing steps at regular intervals to ensure the medical safety of the antibody molecules produced thereby. Accordingly, the support should withstand the physical and chemical conditions employed in the sterilization and washing steps. Although a support not satisfying this requirement may be used, such a support should be frequently replaced by a fresh one in this case, which is highly inefficient economically. Although the covalent bond (isourea bond) generated by the cyanogen bromide according to the above-described invention scarcely causes any troubles under neutral or acidic solution conditions usually employed in affinity chromatography operations, it is cleaved in the presence of an alkaline solution usually employed in sterilizing and washing the support, thereby causing the drop out of ligand. Therefore, the operation conditions in the sterilization and washing steps, which are essentially required particularly in a process of producing a drug (an antibody drugs) with the use of protein A, are severely restricted. In this regard, the above-described invention results in a large progress in the characteristic 1) but still suffers from an industrially serious problem.

As the countermeasures for overcoming the above problems, there have been proposed a method of using a dislufide bond and a method of using a thioether bond (Patent Documents 2 and 3). Owing to advances in gene recombination techniques whereby an arbitrary amino acid sequence in a protein can be modified, these methods each include artificially inserting a cysteine residue into the carboxy end of a protein A molecule, and immobilizing the protein A via a covalent bond at a single site by specifically using a sulfhydryl (SH) group which is a side chain of the cysteine residue. In the former method, a preparation having a sulfhydryl group exposed to the surface (for example, Activated Thiol SEPHAROSE 4B manufactured by Pharmacia Fine Chemicals) is employed as the support and a disulfide bond is formed by the condensation reaction between sulfhydryl groups in both of the recombinant protein A and the support to thereby achieve the site-specific immobilization. In the latter method, a glycopolymer support such as agarose is preliminarily activated with an active epoxy group-introducing reagent such as epichlorohydrin, and a thioether bond is subsequently formed together with the sulfhydryl group of the recombinant protein A to thereby achieve the site-specific immobilization. In each of these methods, protein A can be immobilized at a single site at the carboxy end, which brings about advantages such that the binding stability due to the covalent bond can be ensured and the molecular orientation can be uniformed while sustaining the binding sites of the protein A.

According to these methods, since protein A can be immobilized to the support in a completely uniformly oriented state, most of the immobilized protein A molecules are in the activated form, and therefore, the characteristic 2) as discussed above, namely the amount of the antibody molecules adsorbed by the protein A affinity chromatography support thus constructed, can be largely improved. Furthermore, since the orientation uniformity is maintained, the reversibility of the denaturation of the immobilized protein A molecules can be also elevated. However, although the disulfide bond and the thio ether bond with the use of a sulfhydryl group which is a side chain of a cysteine residue are somewhat improved in durability compared with the isourea bond as described above, they are also cleaved and cause drop out of ligand in the presence of an alkaline solution. Thus, there still remains the serious problem in the sterilization and washing steps.

Patent Document 1: U.S. Pat. No. 3,995,018

Patent Document 2: U.S. Pat. No. 5,084,559 (JP-A-63-267281)

Patent Document 3: U.S. Pat. No. 6,399,750 (JP-T-2000-500649)

Non-Patent Document 1: Forsgren, A. and Sjoquist, J.: J. Immunol. (1966) 97, 822-827

### DISCLOSURE OF THE INVENTION

### Problems that the Invention is to Solve

To achieve a more efficient purification process for antibody molecules, the present inventors considered it important to solve the problem of the binding stability encountering in affinity supports for purifying antibodies having been developed so far, which are typified by the above-described protein A affinity chromatography support being capable of adsorbing a large amount of antibody molecules and characterized by maintaining the orientation uniformity and the problem relating to the sterilization and washing step accompanying the same; and to achieve higher absorbed amount of antibody molecules. Accordingly, an object of the invention is to overcome these problems.

### Means for Solving the Problems

To solve the above-described problems, the inventors have conducted intensive studies and attempted to solve these problems from the following three viewpoints.

The first point is to use, in immobilizing a protein capable of binding to an antibody molecule such as protein A, not a reaction with the use of a side chain as in the conventional cases but an immobilization reaction with the use of a more stable amide (peptide) bond mediated by the main chain.

Concerning a novel immobilization reaction mediated by the main chain of a protein, the present inventors have already developed a method of immobilizing a carboxyl group of the carboxy end of a protein to a support having a primary amine via a peptide (amide) bond with the use of an amide bond-forming reaction mediated by a cyanocysteine residue (Japanese Patent No. 3047020, JP-A-10-45798, Japanese Patent Application No. 2003-106825). According to this method, it is expected that the surface density of the immobilization support can be elevated almost twice as much in the conventional methods. Moreover, the orientation of the protein can be fixed at the carboxyl group at the carboxy end of the main chain via an extremely stable bond, which is the peptide (amide) bond, so that a support being highly tolerant to various physical and chemical treatments while maintaining a high activity in practice can be realized.

Secondly, it is intended to improve a protein capable of binding to an antibody molecule, which is to be used in the immobilization reaction, for fitting in well to the immobilization reaction.

To use the amide bond-forming reaction mediated by a cyanocysteine residue as described above, it is essentially required to introduce an appropriate linker, a cysteine residue and a sequence for efficiently conducting the immobilization reaction into the carboxy end side of the protein.

Natural-origin proteins capable of binding to an antibody molecule such as protein A have a repeated sequence and a high molecular weight of several ten thousands or more. Therefore, the reversibility in denaturation-regeneration thereof can be hardly ensured and use of an autoclave or a potent denaturing agent is restricted in the sterilization and washing steps. Thus, it is essentially required to modify the sequence thereof to achieve the above objects.

In this regard, the inventors have conducted intensive studies and have paid their attention to the fact that a single unit of a repeated structure is capable of binding to an antibody molecule (see, B. Nilsson, et al., Protein Eng., 1, 107-113 (1987)) and that the binding force is elevated twice by using two repeating units but no remarkable effect of elevating the binding force can be established any more by further increasing the repeating units (see, C. Ljungquist, et al., Eur. J. Biochem., 186, 557-561 (1989). Namely, they considered that the above-described problems could be solved by immobilizing a sequence having one or two repeating units and attempted to modify sequences. Consequently, they have successfully found that the above-described problems can be overcome thereby.

Thirdly, the inventors have considered it important to elevate the primary amino group content on an insoluble support in order to bind a larger amount of an antibody-binding protein to the insoluble support by using a primary amine while regulating the orientation. To achieve the above, attempts have been made to introduce a polymer compound (NH₂—X)ₙ having primary amino group into the insoluble support and utilize the same. As a result, they have found that an affinity support, to which a protein capable of binding to an antibody molecule can be immobilized in an increased amount, can be constructed and thus an elevated ability to adsorb an antibody can be established.

As the results of studies made from these three viewpoints, it has been clarified that the above-described problems can be completely overcome according to the invention, thereby completing the present invention.

Accordingly, the present invention relates to the following constitutions.

(1) A support having an affinity for an antibody, which comprises a protein or peptide capable of binding to an antibody molecule,

said protein or peptide being immobilized at a carboxy end thereof to an insoluble support having a primary amino group via an amide bond mediated by a linker sequence.

(2) The support having an affinity for an antibody according to (1), wherein said immobilized insoluble support having a primary amino group comprises a polymer compound having a primary amino group in the repeated structure thereof.

(3) The support having an affinity for an antibody according to (2), wherein the polymer compound having a primary amino group in the repeated structure thereof is polyarylamine.

(4) The support having an affinity for an antibody according to (2), wherein the polymer compound having a primary amino group in the repeated structure thereof is polylysine.

(5) The support having an affinity for an antibody according to any one of (1) to (4), wherein the protein capable of binding to an antibody molecule has an amino acid sequence selected from the group consisting of the amino acid sequences represented by SEQ ID NOs:1 to 4 in Sequence Listing.

(6) The support having an affinity for an antibody according to (1), which is represented by the following formula (1):
NH₂—R₁—CO—NH—R₂—CO—NH—Y (1)

wherein R₁ represents an amino acid sequence of the protein or peptide capable of binding to an antibody molecule; R₂ arbitrarily represents an amino acid sequence of the linker sequence; and Y arbitrarily represents said immobilized support.

(7) The support having an affinity for an antibody according to (6), wherein the moiety represented by CO—NH—R₂—CO in the formula (1) is represented by the following formula (4):
CO—[NH_CH₂—CO]ₘ—CO (4)

wherein m represents a positive integer.

(8) The support having an affinity for an antibody according to (6), wherein the amino acid sequence of the protein capable of binding to an antibody molecule in the definition of the formula (1) is any one of the sequences represented by SEQ ID NOs:1 to 4 in Sequence Listing.

(9) A support for purifying an antibody, which comprises the support having an affinity for an antibody according to any one of (1) to (9).

(10) A method for separating and purifying an antibody molecule, which comprises using the support having an affinity for an antibody according to any one of (1) to (8).

(11) A modified protein binding to an antibody, which is represented by the following formula (2):
NH₂—R₁—CONH—R₂—CO—NH—CH(CH₂—SH)—CO—NH—R₃—COOH (2)

wherein R₁ represents an amino acid sequence of a protein or peptide capable of binding to an antibody molecule; R₂ arbitrarily represents an amino acid sequence of a linker sequence; and R₃ represents an amino acid sequence which is strongly negatively charged around neutrality and is capable of making an isoelectric point of NH₂—R₁—CONH—R₂—CO—NH—CH(CH₂—SH)—CO—NH—R₃—COOH acidic.

(12) The modified protein binding to an antibody according to (11), wherein the moiety represented by NH—R₃—COOH in the formula (2) is represented by the following formula (3):
NH—CH(CH₃)—CO[NH—CH(CH₂—COOH)—CO]ₙ—OH (3)

wherein n represents a positive integer.

(13) The modified protein binding to an antibody according to (11), wherein the moiety represented by CO—NH—R₂—CO in the formula (2) is represented by the following formula (4):
CO—[NH—CH₂—CO]ₘ—CO (4)

wherein m represents a positive integer.

(14) The modified protein binding to an antibody according to (11), wherein the amino acid sequence of the protein capable of binding to an antibody molecule in the formula (2) is any one of the sequences represented by SEQ ID NOs:1 to 4 in Sequence Listing.

### ADVANTAGE OF THE INVENTION

The support, which has modified protein A immobilized thereto, prepared in accordance with the present invention can specifically adsorb antibody molecules in a large amount (almost twice) in comparison with marketed supports for adsorbing antibodies. Accordingly, an extremely-high process efficiency and a high economical efficiency can be achieved by a purification process with the use of the support of the present invention. Moreover, since amide bonds having extremely high chemical and physical stabilities are formed between the modified protein A and the support, it is possible to provide a support capable of withstanding the severe conditions (a high temperature or a treatment with a strong alkali) required in the sterilization and washing steps for applying in a process for producing a drug such as an antibody drug.

### BEST MODE FOR CARRYING OUT THE INVENTION

The present invention provides an affinity support for purifying an antibody in which a protein or peptide capable of binding to an antibody molecule is strongly bound to an insoluble support having a primary amino group via an amide (peptide) bond.

According to the present invention, any protein or peptide may be used so long as it is capable of binding to an antibody molecule to be immobilized. As the support to be used as an insoluble support, any insoluble support having a primary amino group may be used without any restriction with regard to the support type.

1. Insoluble Support to be Immobilized

As the insoluble support having a primary amino group to be used in the present invention, any insoluble support may be used so long as it has a primary amino group. Examples of marketed supports having a primary amino group include AMINO CELLULOFINE (sold by SEIKAGAKU COORPORATION), AF-AMINO TOYOPEARL (sold by TOSOH COORPORATION), EAH-SEPHAROSE 4B and LYSINE-SEPHAROSE 4B (sold by AMERSHAM BIOSCIENCES), PORUS 20NH (sold by BOEHRINGER MANNHEIM) and the like. Further, it is also possible to use glass beads or the like into which a primary amino group has been introduced by using a silane compound having a primary amino group (for example, 3-aminopropylmethoxysilane, etc.).

Furthermore, the primary amino group content per unit volume of the support can be elevated by introducing a polymer compound having the primary amino group in its repeated unit into the insoluble support (see Japanese Patent Application No. 2003-106825).

As a support in which a polymer compound having a primary amino group in its repeated unit is introduced into an insoluble support, for example, polyarylamine-grafted cellulofine is known (see a referential document: Ung-Jin Kim, Shigenori Kuga, Journal of Chromatography A, 946, 283-289 (2002)). Furhter, there are also known CNBr-activated sepharose FF, NHS-activated sepharose FF and a support having a chemical reactivity with a primary amine group. By treating such a support with a polymer compound having a primary amino group in its repeated unit, a support in which the polymer compound is bound to the support via a covalent bond can be prepared. In such a case, the content of the primary amino group usable in the immobilization reaction in the thus prepared support can be varied by appropriately controlling the mixing ratio of the polymer compound having a primary amino group in its repeated unit to the activated support.

As the polymer compound, on the other hand, any polymer compound which has a primary amino group and in which the part other than the primary amino group is substantially inactive with the protein to be immobilized may be used. Among marketed polymer compounds, it is possible to use polyarylamine, poly-L-lysine, and the like.

Accordingly, the present invention is not particularly restricted depending on the type of the immobilization support.

2. Protein Capable of Binding to Antibody Molecule

According to the present invention, the protein or peptide to be immobilized may be an arbitrary one so long as it is capable of binding to an antibody molecule.

Examples of the protein capable of binding to an antibody molecule include protein A derived from Staphylococcus aureus (described in A. Forsgren and J. Sjoequist, J. Immunol. (1966), 97, 822-877), protein G derived from Streptococcus sp. Group C/G (described in EP 0,131,142A2 (1983)), protein L derived from Preptostreptocuccus magnus (described in U.S. Pat. No. 5,965,390 (1992)), protein H derived from group A Streptococcus (described in U.S. Pat. No. 5,180,810 (1993)), protein D derived from Haemophilus influenza (described in U.S. Pat. No. 6,025,484 (1990)), Protein Arp (Protein Arp 4) derived from Streptococcus AP4 (described in U.S. Pat. No. 5,210,183 (1987)), Streptococcal FcRc derived from group C Streptococcus (described in U.S. Pat. No. 4,900,660 (1985)), a protein derived from group A streptococcus, Type II strain (described in U.S. Pat. No. 5,556,944 (1991)), a protein derived from human colonic mucosal epithelial Cells (described in U.S. Pat. No. 6,271,362 (1994)), a protein derived from Staphylococcus aureu, strain 8325-4 (described in U.S. Pat. No. 6,548,639 (1997)), a protein derived from Pseudomonas maltophilia (described in U.S. Pat. No. 5,245,016 (1991)) and the like.

It has been also revealed that many of these proteins have repeated sequences and fragments of these proteins still sustain the capability of binding an antibody molecule. The protein and peptide capable of binding to a target antibody molecule in the present invention include the above antibody-binding proteins derived from natural materials, partial proteins thereof, sequence-modified proteins thereof, partial peptides thereof, mimic peptides thereof, artificial peptides capable of binding to an antibody molecule and the like. These proteins capable of binding to an antibody molecule are represented by the following formula (6)
NH₂—R₁—CO—OH (6)

wherein R₁ represents the amino acid sequence of a peptide or protein capable of binding to an antibody molecule.

According to the present invention, the term “amino acid sequence” as used in defining the formula means an amino acid sequence from which the terminal amino group and the terminal carboxyl group have been removed.

According to the present invention, to enable the immobilization of the protein or peptide represented by the formula (6) NH₂—R₁—CO—OH, it is required to prepare a protein for immobilization represented by the formula (2) NH₂—R₁—CONH—R₂—CO—NH—CH(CH₂—SH)—CO—NH—R₃—COOH. In these formulae, R₃ represents a chain of arbitrary amino acid residues, which is strongly negatively charged around neutrality and is capable of making the isoelectric point of NH₂—R₁—CONH—R₂—CO—NH—CH(CH₂—SH)—CO—NH—R₃—COOH acidic. R₁ represents the amino acid sequence of the above-described protein or peptide capable of binding to an antibody molecule. R₂ represents the amino acid sequence of a linker peptide provided between the protein to be immobilized and represented by the above-described formula (1) and the support. The amino acid sequence R₂ is an arbitrary one without restriction with regard to the type and number thereof. For example, Gly-Gly-Gly-Gly and the like may be used therefor.

Such a fusion protein can be obtained by binding a gene encoding the protein represented by the above-described formula (6) with a gene encoding a peptide sequence represented by the formula (7):
NH₂—R₂—CO—NH—CH(CH₂—SH)—CO—NH—R₃—COOH (7)
wherein R₂ and R₃ are as defined above; to prepare a gene encoding a fusion protein represented by the formula (2) NH₂—R₁—CONH—R₂—CO—NH—CH(CH₂—SH)—CO—NH—R₃—COOH, expressing it in a host such as Escherichia coli, and then separating and purifying the protein thus expressed. The fusion protein can be obtained by using a conventional technique (see, for example, M. Iwakura et al., J. Biochem. 111, 37-45 (1992)). In addition, the above-described fusion protein can be prepared by combining a genetic engineering procedure with a conventional protein synthesis technique or by using such a protein synthesis technique alone.

As R₃ in the above-described formulae (2) and (7), sequences rich in aspartic acid and glutamic acid are preferable. It is more preferable to design a sequence rich in aspartic acid and glutamic acid so as to adjust the isoelectric point of the substance of the formula (2) between 4 and 5. Among these sequences, aranyl-polyaspartic acid can be mentioned as a favorable example thereof. This is because the amide bond-forming reaction mediated by a cyanocysteine residue can easily occur by providing alanine as the amino acid residue following the cyanocysteine residue and the carboxyl group in aspartic acid has the strongest acidity among amino acid side chains.

To further illustrate the above-described matter in greater detail, explanations with employing protein A derived from Staphylococcus aureus as an example will be described.

Protein A derived from Staphylococcus aureus is composed of five domains respectively named A, B, C, D and E having amino acid sequences closely similar to each other and the sequences associated thereto. Each of these domains is constituted from 57 amino acids and has a stable structure by itself. Further, they can be expressed in a large amount in, for example, Escherichia coli. Each domain can exhibit its ability to bind to an antibody molecule by itself. Although the binding force is weaker than the whole protein A derived from natural materials, the binding force of a construct including two domains bound to each other is almost similar to that of the whole protein A derived from natural materials.

In the case of an affinity support for purifying antibody molecules, the minimization of unnecessary sequences largely contributes to simplification of the construction of a recombinant, economical efficiency, control of the binding stability and solution of problems regarding the sterilization and washing steps. Thus, with an attention to the domains of protein A, two types of sequences for immobilization, i.e., a single domain (referred to as a monomer) and two domains bound to each other (referred to as a dimer) are designed.

The amino acid sequence represented by SEQ ID NO:1 in Sequence Listing shows the amino acid sequence of a protein for immobilization, which is prepared for subjecting the A domain monomer of protein A to the immobilization reaction. The amino acid sequence represented by SEQ ID NO:2 therein shows the amino acid sequence of a protein for immobilization, which is prepared for subjecting the A domain dimer of protein A to the immobilization reaction.

SEQ ID NO: 1 Protein for immobilization (A domain
monomer + linker (underlined))
Ala Asp Asn Asn Phe Asn Lys Glu Gln Gln Asn Ala
Phe Tyr Glu Ile Leu Asn Met Pro Asn Leu Asn Glu
Glu Gln Arg Asn Gly Phe Ile Gln Ser Leu Lys Asp
Asp Pro Ser Gln Ser Ala Asn Leu Leu Ala Glu Ala
Lys Lys Leu Asn Glu Ser Gln Ala Pro Lys Gly Gly
Gly Gly Cys Ala Asp Asp Asp Asp Asp Asp
SEQ ID NO: 2 Protein for immobilization (A domain
dimer + linker (underlined))
Ala Asp Asn Asn Phe Asn Lys Glu Gln Gln Asn Ala
Phe Tyr Glu Ile Leu Asn Met Pro Asn Leu Asn Glu
Glu Gln Arg Asn Gly Phe Ile Gln Ser Leu Lys Asp
Asp Pro Ser Gln Ser Ala Asn Leu Leu Ser Glu Ala
Lys Lys Leu Asn Glu Ser Gln Ala Pro Lys Ala Asp
Asn Asn Phe Asn Lys Glu Gln Gln Asn Ala Phe Tyr
Glu Ile Leu Asn Met Pro Asn Leu Asn Glu Glu Gln
Arg Asn Gly Phe Ile Gln Ser Leu Lys Asp Asp Pro
Ser Gln Ser Ala Asn Leu Leu Ser Glu Ala Lys Lys
Leu Asn Glu Ser Gln Ala Pro Lys Gly Gly Gly Gly
Cys Ala Asp Asp Asp Asp Asp Asp

The above-described sequences of SEQ ID NOS:1 and 2 are sequences in which a sequence represented by SEQ ID NO:5, which is a sequence of polyglycine-cysteine residue-alanine residue-polyaspartic acid, is added respectively to the carboxy end side of the A domain monomer sequence of protein A and the A domain dimer sequence of protein A represented by the following SEQ ID NOS:3 and 4.

SEQ ID NO: 3 A domain monomer
Ala Asp Asn Asn Phe Asn Lys Glu Gln Gln Asn Ala
Phe Tyr Glu Ile Leu Asn Met Pro Asn Leu Asn Glu
Glu Gln Arg Asn Gly Phe Ile Gln Ser Leu Lys Asp
Asp Pro Ser Gln Ser Ala Asn Leu Leu Ala Glu Ala
Lys Lys Leu Asn Glu Ser Gln Ala Pro Lys
SEQ ID NO: 4 A domain dimer
Ala Asp Asn Asn Phe Asn Lys Glu Gln Gln Asn Ala
Phe Tyr Glu Ile Leu Asn Met Pro Asn Leu Asn Glu
Glu Gln Arg Asn Gly Phe Ile Gln Ser Leu Lys Asp
Asp Pro Ser Gln Ser Ala Asn Leu Leu Ser Glu Ala
Lys Lys Leu Asn Glu Ser Gln Ala Pro Lys Ala Asp
Asn Asn Phe Asn Lys Glu Gln Gln Asn Ala Phe Tyr
Glu Ile Leu Asn Met Pro Asn Leu Asn Glu Glu Gln
Arg Asn Gly Phe Ile Gln Ser Leu Lys Asp Asp Pro
Ser Gln Ser Ala Asn Leu Leu Ser Glu Ala Lys Lys
Leu Asn Glu Ser Gln Ala Pro Lys Gly Gly Gly Gly
Cys Ala Asp Asp Asp Asp Asp Asp
SEQ ID NO: 5 Linker
Gly Gly Gly Gly Cys Ala Asp Asp Asp Asp Asp Asp

Although four glycine residues are shown as the sequence of the linker moiety according to the above-described SEQ ID NO:5, the linker sequence may be an arbitrary one without restriction in the length or type thereof. A cysteine residue is a mandatory residue since it is to be converted into cyanocysteine by cyaniding the SH group in its side chain and used in the immobilization reaction. The subsequent alanine-polyaspartic acid is introduced in order to promote the immobilization reaction and elevate the reaction efficiency. Any sequence may be used therefor, so long as it can adjust the isoelectric points of the proteins represented by SEQ ID NO:1 and SEQ ID NO:2 between 4 and 5.

The proteins represented by SEQ ID NO:1 and SEQ ID NO:2 can be prepared by using chemical synthesis techniques. In addition, they can be prepared by expressing DNAs encoding the amino acid sequences of these proteins in a host such as Escherichia coli and then separating and purifying from the expressing cells.

As the base sequences of DNAs respectively encoding the proteins represented by SEQ ID NO:1 and SEQ ID NO:2, the base sequences represented by SEQ ID NO:6 and SEQ ID NO:7 can be mentioned.

SEQ ID NO: 6 DNA encoding protein for immobiliza-
tion of SEQ ID NO: 1
ATGGCTGATAACAATTTCAACAAAGAACAACAAAATGCTTTCTATGAAAT
CTTGAATATGCCTAACTTAAACGAAGAACAACGCAATGGTTTCATCCAAA
GCTTAAAAGATGACCCAAGCCAAAGTGCTAACCTATTGTCAGAAGCTAAA
AAGTTAAATGAATCTCAAGCACCGAAAGGTGGCGGTGGCTGCGCTGATGA
CGATGACGATGACTAA
SEQ ID NO: 7 DNA encoding protein for immobiliza-
tion of SEQ ID NO: 2
ATGGCTGATAACAATTTCAACAAAGAACAACAAAATGCTTTCTATGAAAT
CTTGAATATGCCTAACTTAAACGAAGAACAACGCAATGGTTTCATCCAAA
GCTTAAAAGATGACCCAAGCCAAAGTGCTAACCTATTGTCAGAAGCTAAA
AAGTTAAATGAATCTCAAGCACCGAAAGCTGATAACAATTTCAACAAAGA
ACAACAAAATGCTTTCTATGAAATCTTGAATATGCCTAACTTAAACGAAG
AACAACGCAATGGTTTCATCCAAAGCTTAAAAGATGACCCAAGCCAAAGT
GCTAACCTATTGTCAGAAGCTAAAAAGTTAAATGAATCTCAAGCACCGAA
AGGTGGCGGTGGCTGCGCTGATGACGATGACGATGACTAA

These base sequences represent sequences having an initiation codon ATG and a termination codon TAA respectively at the 5′ end and the 3′ end.

Since an amino acid-encoding base sequence is degenerated, a plural number of codons correspond to a single amino acid residue. Therefore, the sequences encoding the proteins represented by SEQ ID NO:1 and SEQ ID NO:2 are not restricted to the sequences represented by SEQ ID NO:6 and SEQ ID NO:7 but are present in the number of potential codon combinations.

To express the gene sequences encoding the proteins represented by SEQ ID NO:1 and SEQ ID NO:2 in host cells such as Escherichia coli, the sequences (underlined) required in the transcription and translation of the genes should be added to the upstream of the sequences encoding the proteins. Examples of the gene sequences having such a sequence added thereto to enable the transfer into a vector include the sequences represented by SEQ ID NO:8 and SEQ ID NO:9.

SEQ ID NO: 8 DNA for transferring into vector
(corresponding to DNA of SEQ ID NO: 6)
GGATCCTTGACAATATCTTAACTATCTGTTATAATATATTGACCAGGTTA
ACTAACTAAGCAGCAAAAGGAGGAACGACTATGGCTGATAACAATTTCAA
CAAAGAACAACAAAATGCTTTCTATGAAATCTTGAATATGCCTAACTTAA
ACGAAGAACAACGCAATGGTTTCATCCAAAGCTTAAAAGATGACCCAAGC
CAAAGTGCTAACCTATTGTCAGAAGCTAAAAAGTTAAATGAATCTCAAGC
ACCGAAAGGTGGCGGTGGCTGCGCTGATGACGATGACGATGACTAAGAAT
TC
SEQ ID NO: 9 DNA for transferring into vector
(corresponding to DNA of SEQ ID NO: 7)
GGATCCTTGACAATATCTTAACTATCTGTTATAATATATTGACCAGGTTA
ACTAACTAAGCAGCAAAAGGAGGAACGACTATGGCTGATAACAATTTCAA
CAAAGAACAACAAAATGCTTTCTATGAAATCTTGAATATGCCTAACTTAA
ACGAAGAACAACGCAATGGTTTCATCCAAAGCTTAAAAGATGACCCAAGC
CAAAGTGCTAACCTATTGTCAGAAGCTAAAAAGTTAAATGAATCTCAAGC
ACCGAAAGCTGATAACAATTTCAACAAAGAACAACAAAATGCTTTCTATG
AAATCTTGAATATGCCTAACTTAAACGAAGAACAACGCAATGGTTTCATC
CAAAGCTTAAAAGATGACCCAAGCCAAAGTGCTAACCTATTGTCAGAAGC
TAAAAAGTTAAATGAATCTCAAGCACCGAAAGGTGGCGGTGGCTGCGCTG
ATGACGATGACGATGACTAAGAATTC

The sequences represented by SEQ ID NO:8 and SEQ ID NO:9 are obtained by attaching the following sequence:

SEQ ID NO: 10

TTGACAATATCTTAACTATCTGTTATAATATATTGACCAGGTTAACTAAC
TAAGCAGCAAAAGGAGGAACGACT

to the upstream of the initiation codon respectively in the sequences represented by SEQ ID NO:6 and SEQ ID NO:7, and attaching a cleavage site (GGATCC) recognized by a restriction enzyme BamHI to the 5′ end and another cleavage site (GAATTC) recognized by a restriction enzyme EcoRI to the 3′ end, thereby enabling transfer into a vector.

The sequences represented by SEQ ID NO:8 and SEQ ID NO:9 can be artificially synthesized by chemically synthesizing several fragments and subsequently conducting the PCR method or using enzymes such as DNA ligase.

The synthetic gene thus obtained is integrated into an appropriate vector with the use of the restriction enzyme sites and then expressed in host cells. As the vector, any genes may be used so long as the appropriate restriction enzyme sites can be used therein. In marketed vectors, for example, high copy number vectors of the pUC-series and PBR-series are suitable therefor. By expressing the recombinants having the DNAs represented by SEQ ID NO:6 and SEQ ID NO:7, the proteins represented by SEQ ID NO:1 and SEQ ID NO:2 in a soluble state can be expressed and accumulated up to an extent of from 5 to 30% of the somatic proteins in the case of, for example, Escherichia coli.

The protein thus expressed and accumulated can be homogeneously purified from a cell-free extract of the expressing cells by a chromatographic operation commonly employed in purifying proteins. As the chromatography usable therefor, anion exchange chromatography, gel filtration chromatography and the like can be effectively employed. Among all, affinity chromatography with the use of a support having immunoglobulin immobilized thereto is most effective, since it is capable of binding to an antibody.

3. Immobilization of Protein

According to the present invention, an amide bond is formed between the carboxyl group at the carboxy end of a protein and the primary amino group carried by an insoluble support with the use of a transamination reaction mediated by cyanocysteine.

Namely, according to the present invention, a modified protein binding to an antibody, which is represented by the formula (2):
NH₂—R₁—CONH—R₂—CO—NH—CH(CH₂—SH)—CO—NH—R₃—COOH (2)
in which R₁ represents the amino acid sequence of a protein or peptide capable of binding to an antibody molecule; R₂ arbitrarily represents the amino acid sequence of a linker sequence; and R₃ represents an amino acid sequence which is strongly negatively charged around neutrality and is capable of making the isoelectric point of NH₂—R₁—CONH—R₂—CO—NH—CH(CH₂—SH)—CO—NH—R₃—COOH acidic; is used to thereby bound a modified protein binding to an antibody, which is represented by the formula (8):
NH₂—R₁—CO—NH—R₂—CO—OH
in which R₁ represents the amino acid sequence of a protein or peptide capable of binding to an antibody molecule; and
R₂ arbitrarily represents the amino acid sequence of a linker sequence;
at one site of the carboxy end to an insoluble support. Therefore, it is necessary to convert a sulfhydryl group of the cysteine residue in the modified protein binding to an antibody, which is represented by formula (2), into cyanocysteine by cyanidation. This conversion can be conducted either before the adsorption of the protein by the support, after the adsorption of the protein by the support or simultaneously with the adsorption.

The cyanidation reaction can be carried out by using a marketed cyanidation reagent. Usually, it is convenient to employ 2-nitro-5-thiocyanobennzoic acid (NTCB) (see, Y. Degani, A. Ptchornik, Biochemistry, 13, 1-11 (1974)), 11-cyano-4-dimethylaminopyridinium tetrafluoroborate (CDAP) or the like. Cyanidation with the use of NTCB can be effectively carried out in a 10 mM phosphate buffer solution having a pH of 7.0. The immobilization reaction proceeds by making the solvent weak alkaline after the completion of the cyanidation reaction. Namely, an amide bond is formed between the carboxyl group of the amino acid residue immediately before the cyanocysteine residue and the primary amine in the support. This can be performed by, for example, replacing the buffer solution with a 10 mM borate buffer solution having a pH of 9.5.

The reaction in which cyanocysteine employed in the present invention is participated is accompanied by side reactions such as a hydrolysis reaction. However, the immobilization reaction efficiency can be elevated to about 80% or higher by using the modified protein represented by the formula (2) as described above, namely, lowering the isoelectric point of the modified protein to pH 4 to 5 by the introduction of R₃ in the formula, to thereby cause rapid ion adsorption due to the interaction between the support and the ion. Furthermore, since the reaction products formed by the side reactions such as the hydrolysis reaction, which accompanies the immobilization reaction mediated by cyanocysteiene, are all soluble in the solvent, these by-products can be removed by washing off the immobilization support with an appropriate solvent after the completion of the reaction.

Therefore, in the support having an affinity for an antibody prepared by the immobilization reaction employed in the present invention, the carboxy end of a protein or peptide capable of binding to an antibody molecule is immobilized via an amide bond mediated by a linker sequence to an insoluble support having a primary amino group and it is represented by the following formula (1):
NH₂—R₁—CO—NH—R₂—CO—NH—Y (1)
in which R₁ represents the amino acid sequence of the protein or peptide capable of binding to an antibody molecule; R₂ arbitrarily represents the amino acid sequence of a linker sequence; and Y arbitrarily represents an immobilization support.

In the support thus prepared, the protein capable of binding to the target antibody molecule is uniformly bound to the support under the regulation of the orientation thereof at a single site of the carboxy end.

4. Utilization as Affinity Support

The thus obtained affinity support represented by the above-described formula (1), which has the protein capable of binding to an antibody molecule immobilized thereto, can be used, for example, for separating and purifying an antibody.

As one of the factors required for an affinity support, the amount of immunoglobulin bound per unit weight or volume of the support may be mentioned. According to the affinity support represented by the formula (1) obtainable by the present invention, since the immobilized protein or peptide capable of binding to an antibody molecule can fully sustain its functions, the above-described factor depends on the number of the protein or peptide molecules capable of binding to the antibody that have been introduced into the support. As will be shown in the EXAMPLES, about 90 mg of immunoglobulin G per ml of the affinity support can be bound and collected by introducing the molecules of the protein or peptide capable of binding to the antibody molecule in the almost maximum number. The maximum binding level of affinity supports for separating and purifying an antibody marketed today is about 50 mg per ml of the affinity support. Accordingly, it is indicated that the affinity support of the present invention is excellent, since an increase in the binding level by about 40 mg/ml can be established.

The affinity support obtainable by the present invention can be used as a chromatogram medium as follows. Namely, a preparation containing immunoglobulin as an antibody is introduced into a column or the like packed with the affinity support of the present invention under neutral conditions. After sufficiently washing with a neutral buffer solution containing a salt (NaCl, KCl, etc.) at a high concentration, elution was carried out with an appropriate buffer solution having pH of 3 to 5. Then, immunoglobin can be homogenously separated and purified. Although the separation conditions depend on the properties of the target immunoglobulin, homogenous immunoglobulin can be obtained at a yield of 100% by optimizing the separation conditions.

So long as the insoluble support having a primary amino group employed in the preparation is stable to heat treatments, the affinity support of the present invention can be subjected to a sterilization treatment at a high temperature by using an autoclave, steam or the like to such an extent as not cleaving peptide bonds. As a result, it becomes possible to simplify the sterilization and cleaning treatments for the whole process of purifying immunoglobulin, which is appropriate for the production process of an immunoglobulin preparation to be used as a pharmaceutical.

Next, the present invention will be described by referring to the following EXAMPLES. However, the invention is not restricted thereto.

As insoluble supports in the following EXAMPLES, a support prepared by treating a marketed insoluble support CNBr-activated SEPHAROSE (purchased from PHARMACIA) with a marketed L-polyarylamine (marketed from NITTO BOSEKI Co., Ltd.) for binding and marketed AMINO-CELLULOFINE (marketed from SEIKAGAKU COORPORATION) were employed.

As the protein corresponding to the formula (2), the modified proteins binding to antibody and represented by Sequence Listings 1 and 2 were employed in the EXAMPLES.

### EXAMPLE 1

### Construction of Polyarylamine-Bound Sepharose

Five g of CNBr-activated sepharose was suspended in 20 ml of 1 mM hydrochloric acid. After allowing for swelling for 30 minutes, it was washed with 50 ml of 1 mM hydrochloric acid. The insoluble matters were collected, suspended in 20 ml of a 0.1% solution of L-polyarylamine and gently mixed for 12 hours, thereby conducting the binding reaction. Then, the insoluble matters were suspended in 20 ml of a 1 M monoethanolamine solution and gently stirred at room temperature for 4 hours to thereby mask unreacted active groups on the support. Further, washing with 20 ml portions of a 50 mM glycine/HCl buffer solution (pH 3.5) containing 1 M of NaCl and washing with 20 ml portions of a 50 mM Tris/HCl buffer solution (pH 8.0) containing 1 M of NaCl were alternately repeated 8 times. The insoluble matters thus obtained were collected and employed in the subsequent protein immobilization.

The content of the primary amine having been introduced into the polyarylamine-bound sepharose thus obtained was determined by a coloration reaction (R. Fields, Methods in Enzymology, 25, p. 464-468 (1971)). As a result, it showed obviously stronger coloration than marketed supports containing primary amine such as AMINO CELLULOFINE (sold by SEIKAGAKU COORPORATION), AF-AMINO TOYOPEARL (sold by TOSOH COORPORATION), EAH-SEPHAROSE 4B and LYSINE-SEPHAROSE 4B (sold by AMERSHAM BIOSCIENCES), AFFIGEL (sold by BIO RAD) and PORUS 20NH (sold by BOEHRINGER MANNHEIM), thereby indicating a high primary amine content.

### EXAMPLE 2

### Preparation of Modified Protein Binding to Antibody

As antibody-binding proteins, a product obtained by modifying the A domain monomer of protein A derived from Staphylococcus aureus and a dimer having two monomers bound to each other was used. The amino acid sequences of the modified protein derived from the monomer and the modified protein derived from the dimer are respectively represented by SEQ ID NO:1 and SEQ ID NO:2.

As the gene sequences capable of expressing the modified proteins which bind to antibody and are respectively represented by SEQ ID NO:1 and SEQ ID NO:2, DNA sequences respectively represented by SEQ ID NO:8 and SEQ ID NO:9 were designed. Based on the sequences thus designed, artificially synthesized genes were prepared by carrying our fragmentary chemical synthesis in combination with the PCR method, fragment binding with the use of a DNA ligase, and the like. These artificially synthesized genes had restriction sites BamHI and EcoRI having been introduced at the terminal parts. Using these sites, they were transferred into the BamHI and EcoRI sites of an expression vector pUC18 and transduced into Escherichia coli JM109 strain. From the transformants thus obtained, recombinant plasmids were separated and the base sequence of the parts located between the BamHI and EcoRI sites was examined. Then, recombinant plasmids into which the sequences represented by SEQ ID NO:8 and SEQ ID NO:9 had been exactly integrated were selected and named PAA2 and PAAD1, respectively. Each of the separated PAA2 and PAAD1 was transduced again into Escherichia coli JM109 strain and then cultured in 2 l of a medium (containing 20 g of sodium chloride, 20 g of yeast extract, 32 g of trypton and 100 mg of ampicillin sodium) at 37° C. overnight. Subsequently, the liquid culture medium was centrifuged at a low speed (5000 rpm per minute) to thereby obtain about 5 g of moist cells.

These cells were suspended in 40 ml of a 10 mM phosphate buffer solution (pH 7.0) containing 1 mM of ethylenediaminetetraacetic acid (EDTA) (buffer 1). After disrupting the cells by a French press, the mixture was centrifuged for 20 minutes (20,000 rpm per minite) and the supernatant was separated. To the obtained supernatant, streptomycin sulfate was added to give a final concentration of 2%. After stirring at 4° C. for 20 minutes, it was centrifuged for 20 minutes (20,000 rpm per minute) and the supernatant was separated. To the obtained supernatant, ammonium sulfate was added to give a final concentration of 40%. After stirring at 4° C. for 20 minutes, it was centrifuged for 20 minutes (20,000 rpm per minute) and the supernatant was separated.

Then, it was applied to a column (10 ml) packed with an IgG SEPHAROSE 6 fast flow (purchased from AMERSHAM BIOSCIENCES) equilibrated with the buffer 1. After passing through 100 ml of the buffer 1, 500 ml of the buffer 1 containing 0.5 M of KCl was passed through. After confirming that no protein was contained in the eluate, 100 ml of distilled water was passed to thereby remove salts. Next, the modified protein binding to antibody, which bound to the column, was eluted with 100 ml of 0.1 M acetic acid. The eluate was collected in 2 ml portions by using a fraction collector and about 16 ml of protein-elution fractions were collected. As a result, about 10 mg and about 16 mg of purified preparations of the modified proteins binding to antibody, which are respectively represented by SEQ ID NO:1 and SEQ ID NO:2, were obtained. The protein fractions thus obtained were dried with the use of a vacuum dryer, thereby conducting an concentration together with a removal of acetic acid. Even after such a purification treatment, the antibody-binding abilities were completely sustained. The dry preparations thus obtained were dissolved in an appropriate buffer solution and employed in the immobilization reaction.

### EXAMPLE 3

### Immobilization of Protein

The dry preparations of the antibody-binding proteins respectively represented by SEQ ID NO:1 and SEQ ID NO:2 obtained in EXAMPLE 3 were first dissolved in a 10 mM phosphate buffer solution (pH 7.0) containing 5 mM of ethylenediaminetetraacetic acid (EDTA) (buffer 2) to give a concentration of 1 mg/ml. Then, the obtained solutions were appropriately diluted with the buffer 2 to give protein preparations having various concentrations.

990 μl of each of the preparations of the antibody-binding proteins respectively represented by SEQ ID NO:1 and SEQ ID NO:2, which had been adjusted at various concentrations, was mixed with 10 μl of the polyarylamine-bound sepharose prepared in EXAMPLE 1 and gently stirred at room temperature for 2 hours or longer. Next, it was centrifuged at 1,000 rpm for several seconds and the insoluble matters were collected (Step 1). The insoluble matters were suspended in the buffer 2 containing 5 mM of 2-nitro-5-thiocyanobensoic acid (NTCB) and a cyanidation reaction was conducted while gently stirring the mixture at room temperature for 4 hours (Step 2). Then, the reaction mixture was washed with 1 ml portions of the buffer 2 five times. The insoluble matters were suspended in 1 ml of a 10 mM borate buffer solution (pH 9.5) containing 5 mM of EDTA and an immobilization reaction was conducted while gently stirring the mixture at room temperature for 24 hours (Step 3). Subsequently, the insoluble matters were washed with 1 ml portions of a 10 mM buffer solution (pH 7.0) containing 1 M of KCl five times to thereby remove the unreacted matters and by-products of the immobilization reaction (Step).

The amount of the protein immobilized to the polyarylamine-bound sepharose was determined by quantifying the protein in the solution employed in each step of the immobilization reaction and subtracting the amount of the protein contained in the recovered solution from the total protein amount subjected to the reaction. The amount of the immobilized protein was increased with an increase in the amount of the protein added. The maximum immobilization level was achieved at the point when the adsorption due to the static interaction attained the maximum in Step 1. Both of the antibody-binding proteins respectively represented by SEQ ID NO:1 and SEQ ID NO:2 were immobilized at a ratio of about 11 nM protein per 10 μl of the polyarylamine-bound sepharose.

The protein concentrations in the preparations of the antibody-binding proteins respectively represented by SEQ ID NO:1 and SEQ ID NO:2 were determined by measuring the absorbances at 224 nm and 233.3 nm (W. E. Groves, et. al., Anal. Biochem., 22, 195-210 (1968)).

### EXAMPLE 4

### Measurement of Binding Force of Support Having Protein of Antibody-Binding Protein Preparation Immobilized Thereto to Immunoglobulin G

The binding abilities of the supports having the proteins of the antibody-protein preparations immobilized thereto, which was prepared in EXAMPLE 3, to an antibody molecule were measured in the following manner.

A 10 μl portion of each of the supports respectively having the antibody-binding proteins respectively represented by SEQ ID NO:1 and SEQ ID NO:2 immobilized thereto was mixed with 990 μl of human-origin immunoglobulin (2 mg) in a 10 mM phosphate buffer (pH 7.0). After gently stirring at room temperature for 12 hours, the mixture was washed with 1 ml portions of 10 mM phosphate buffer (pH 7.0) containing 1 M of KCl five times. By measuring the absorbance at 280 nm, it was confirmed that the final washing liquor contained no protein.

The immunoglobulin G was separated from the support by adding 1 ml of a 0.1 M acetic acid solution to the insoluble support collected by centrifugation after the washing. The amount of immunoglobulin released in the solution was determined by measuring the absorbance at 280 nm and using the coefficient of absorbance (E₂₈₀^{1%}=14.0). As a result, it was found out that 430 μg and 890 μg of the antibody-binding proteins respectively represented by Sequence Listing 1 and Sequence Listing 2 were eluted respectively from the supports having these proteins immobilized thereto.

For comparison, two affinity supports having protein A immobilized thereto, which showed the highest antibody-binding abilities from among marketed ones, were purchased and the immobilized and released immunoglobulin G was quantified by the above-described method. The results are shown in Table 1.

TABLE 1
Comparison of human immunoglobulin-binding abilities of
affinity supports having antibody-binding protein
immobilized thereto

| | Amount of |
|---|---|
| | immobilized/released |
| | immunoglobulin G (mg/ml |
| Immobilization support | support) |
| Support having antibody-binding | ca. 63 |
| protein of SEQ ID NO: 1 | |
| immobilized thereto | |
| Support having antibody-binding | ca. 89 |
| protein of SEQ ID NO: 1 | |
| immobilized thereto | |
| Marketed product 1 | ca. 46 (50*) |
| Marketed product 2 | ca. 31 (35*) |

*The antibody-adsorption capacity indicated in the catalog of a marketed support for adsorbing an antibody.

According to the immobilization supports of the present invention, the support having the antibody-binding protein represented by SEQ ID NO:2 immobilized thereto shows an adsorption ability about twice as much as the adsorption ability (50 mg/ml support) of the marketed support that shows the highest ability among marketed supports for adsorbing an antibody, which proves that the present invention is excellent.

1. A support having an affinity for an antibody, which comprises a protein or peptide capable of binding to an antibody molecule,
said protein or peptide being immobilized at a carboxy end thereof to an insoluble support having a primary amino group via an amide bond mediated by a linker sequence.

2. The support having an affinity for an antibody according to claim 1, wherein said immobilized insoluble support having a primary amino group comprises a polymer compound having a primary amino group in the repeated structure thereof.

3. The support having an affinity for an antibody according to claim 2, wherein the polymer compound having a primary amino group in the repeated structure thereof is polyarylamine.

4. The support having an affinity for an antibody according to claim 2, wherein the polymer compound having a primary amino group in the repeated structure thereof is polylysine.

5. The support having an affinity for an antibody according to claim 1, wherein the protein capable of binding to an antibody molecule has an amino acid sequence selected from the group consisting of the amino acid sequences represented by SEQ ID NOs:1 to 4 in Sequence Listing.

6. The support having an affinity for an antibody according to claim 1, which is represented by the following formula (1):
NH₂—R₁—CO—NH—R₂—CO—NH—Y (1)
wherein R₁ represents an amino acid sequence of the protein or peptide capable of binding to an antibody molecule; R₂ arbitrarily represents an amino acid sequence of the linker sequence; and Y arbitrarily represents said immobilized support.

7. The support having an affinity for an antibody according to claim 6, wherein the moiety represented by CO—NH—R₂—CO in the formula (1) is represented by the following formula (4):
CO—[NH—CH₂—CO]ₘ—CO (4)
wherein in represents a positive integer.

8. The support having an affinity for an antibody according to claim 6, wherein the amino acid sequence of the protein capable of binding to an antibody molecule in the definition of the formula (1) is any one of the sequences represented by SEQ ID NOs:1 to 4 in Sequence Listing.

9. A support for purifying an antibody, which comprises the support having an affinity for an antibody according to claim 1.

10. A method for separating and purifying an antibody molecule, which comprises using the support having an affinity for an antibody according to claim 1.

11. A modified protein binding to an antibody, which is represented by the following formula (2):
NH₂—R₁—CONH—R₂—CO—NH—CH(CH₂—SH)—CO—NH—R₃—COOH (2)
wherein R₁ represents an amino acid sequence of a protein or peptide capable of binding to an antibody molecule; R₂ arbitrarily represents an amino acid sequence of a linker sequence; and R₃ represents an amino acid sequence which is strongly negatively charged around neutrality and is capable of making an isoelectric point of NH₂—R₁—CONH—R₂—CO—NH—CH(CH₂—SH)—CO—NH—R₃—COOH acidic.

12. The modified protein binding to an antibody according to claim 11, wherein the moiety represented by NH—R₃—COOH in the formula (2) is represented by the following formula (3):
NH—CH(CH₃)—CO[NH—CH(CH₂—COOH)—CO]ₙ—OH (3)
wherein n represents a positive integer.

13. The modified protein binding to an antibody according to claim 11, wherein the moiety represented by CO—NH—R₂—CO in the formula (2) is represented by the following formula (4):
CO—[NH—CH₂—CO]ₘ—CO (4)
wherein in represents a positive integer.

14. The modified protein binding to an antibody according to claim 11, wherein the amino acid sequence of the protein capable of binding to an antibody molecule in the formula (2) is any one of the sequences represented by SEQ ID NOs:1 to 4 in Sequence Listing.


SEQUENCE LISTING (10 sequences)

SEQ ID NO: 1 - protein, 70 aa, Artificial sequence
  Synthetic protein for antibody immobilization
ADNNFNKEQQNAFYEILNMPNLNEEQRNGFIQSLKDDPSQSANLLAEAKKLNESQAPKGGGGCADDDDDD

SEQ ID NO: 2 - protein, 128 aa, Artificial Sequence
  Synthetic protein for antibody immobilization
ADNNFNKEQQNAFYEILNMPNLNEEQRNGFIQSLKDDPSQSANLLSEAKKLNESQAPKADNNFNKEQQNAFYEILNMPNLNEEQRNGFIQSLKDDPSQSANLLSEAKKLNESQAPKGGGGCADDDDDD

SEQ ID NO: 3 - protein, 58 aa, Artificial sequence
  A domain monomer from Staphylococcus aureus
ADNNFNKEQQNAFYEILNMPNLNEEQRNGFIQSLKDDPSQSANLLAEAKKLNESQAPK

SEQ ID NO: 4 - protein, 128 aa, Artificial Sequence; identical to SEQ ID NO: 2
  A domain dimer from Staphylococcus aureus

SEQ ID NO: 5 - protein, 12 aa, Artificial Sequence
  Synthetic linker peptide
GGGGCADDDDDD

SEQ ID NO: 6 - DNA, 216 bp, Artificial Sequence
  Synthetic DNA encoding protein for antibody immobilization
atggctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaaggtggcggtggctgcgctgatgacgatgacgatgactaa

SEQ ID NO: 7 - DNA, 390 bp, Artificial Sequence
  Synthetic DNA encoding protein for antibody immobilization
atggctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaaggtggcggtggctgcgctgatgacgatgacgatgactaa

SEQ ID NO: 8 - DNA, 302 bp, Artificial Sequence
  Synthetic DNA for transferring into vector
ggatccttgacaatatcttaactatctgttataatatattgaccaggttaactaactaagcagcaaaaggaggaacgactatggctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaaggtggcggtggctgcgctgatgacgatgacgatgactaagaattc

SEQ ID NO: 9 - DNA, 476 bp, Artificial Sequence
  Synthetic DNA for transferring into vector
ggatccttgacaatatcttaactatctgttataatatattgaccaggttaactaactaagcagcaaaaggaggaacgactatggctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaaggtggcggtggctgcgctgatgacgatgacgatgactaagaattc

SEQ ID NO: 10 - DNA, 74 bp, Artificial Sequence
  Synthetic DNA sequence for gene expression
ttgacaatatcttaactatctgttataatatattgaccaggttaactaactaagcagcaaaaggaggaacgact